(19) United States
(12) Patent Application Publication
(10) Pub. No.: US 2008/0103176 A1
(43) Pub. Date: May 1, 2008

(54) Substituted Hydroxyethylamines

(75) Inventors: Brian S. Bronk, Gales Ferry, CT (US); Todd W. Butler, Salem, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc.

(21) Appl. No.: 11/815,034

(22) PCT Filed: Feb. 2, 2006
(86) PCT No.: PCT/IB06/00301

§ 371 Date: Jul. 30, 2007

Related U.S. Application Data
(60) Provisional application No. 60/653,072, filed on Feb. 14, 2005.

(51) Int. Cl. A61K 31/445; A61P 25/28; C07D 211/06

(52) U.S. Cl. 514/319; 546/205

(57) ABSTRACT

This invention is directed to compounds of the formula (I), as defined herein. The compound of formula I may be used, for example, to treat a disorder or condition that may be treated by inhibiting 13-secretase.

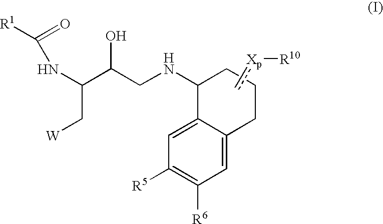

### BACKGROUND OF THE INVENTION

This invention is directed to compounds of formula I described herein, to pharmaceutical composition comprising such compounds, and to methods of treatment using such compounds of disorders or conditions described herein, including Alzheimer's disease.

Alzheimer's disease (AD) is a progressive degenerative disease of the brain primarily associated with aging. AD is characterized by loss of memory, cognition, reasoning, judgment, and orientation. As the disease progresses, motor, sensory, and linguistic abilities are also affected until there is global impairment of multiple cognitive functions. These cognitive losses occur gradually, but typically lead to severe impairment and eventual death in the range of four to twelve years.

Alzheimer's disease is characterized by two major pathologic observations in the brain: neurofibrillary tangles and beta amyloid (or neuritic) plaques, comprised predominantly of an aggregate of a peptide fragment known as A beta peptide or A beta, and referred to herein as A beta or Aβ. Individuals with AD exhibit characteristic A beta deposits in the brain (beta-amyloid plaques) and in cerebral blood vessels (beta-amyloid angiopathy) as well as neurofibrillary tangles. Neurofibrillary tangles occur not only in Alzheimer's disease but also in other dementia-inducing disorders. On autopsy, large numbers of these lesions are generally found in areas of the human brain important for memory and cognition. Smaller numbers of these lesions in a more restricted anatomical distribution are found in the brains of most aged humans who do not have clinical AD. Amyloidogenic plaques and vascular amyloid angiopathy also characterize the brains of individuals with Trisomy (Down's Syndrome), Hereditary Cerebral Hemorrhage with Amyloidosis of the Dutch-Type, and other neurodegenerative disorders. Beta-amyloid is believed to be a causative, precursor or factor in the development of AD. Deposition of beta-amyloid in areas of the brain responsible for cognitive activities is believed to be an important factor in the development of AD. Beta-amyloid plaques are predominantly composed of A beta (also sometimes designated beta A), which is derived by proteolysis of the amyloid precursor protein (APP) and which comprises 39-42 amino acids. Several proteases called secretases are involved in the processing of APP.

Cleavage of APP at the N-terminus of A beta by beta-secretase and at the C-terminus by one or more gamma secretases constitutes the beta-amyloidogenic pathway, i.e. the pathway by which A beta is formed. Cleavage of APP by alpha-secretase produces alpha-sAPP, a secreted form of APP I that does not result in beta-amyloid plaque formation. This alternate pathway precludes the formation of A beta. A description of the proteolytic processing fragments of APP is found, for example, in U.S. Pat. Nos. 5,441,870; 25 5,721,130; and 5,942,400.

An aspartyl protease has been identified as the enzyme responsible for processing of APP at the beta-secretase cleavage site. The beta-secretase enzyme has been disclosed using varied nomenclature, including BACE, Asp, and Memapsin. See, for example, Sinha et al., 1999, Nature 402:537-554 (p 501) and published PCT application WO00/17369.

Several lines of evidence indicate that progressive cerebral deposition of A beta plays a seminal role in the pathogenesis of AD and can precede cognitive symptoms by years or decades. See, e.g., Selkoe, 1991, Neuron 6:487. Release of A beta from neuronal cells grown in culture and the presence of A beta in cerebrospinal fluid (CSF) of both normal individuals and AD patients has been demonstrated. See, for example, Seubert et 5 al., 1992, Nature 359:325-327. It has been proposed that A beta accumulates as a result of APP processing by beta-secretase, thus inhibition of this enzyme's activity is desirable for the treatment of AD.

In vivo processing of APP at the beta-secretase cleavage site is thought to be a rate-limiting step in A beta production, and is thus a therapeutic target for the treatment of AD. See for example, Sabbagh, M., et al., 1997, Alz. Dis. Rev. 3, 1 19. BACE1 knockout mice fail to produce A beta, and present a normal phenotype. When crossed with transgenic mice that overexpress APP, the progeny show reduced amounts of A beta in brain extracts as compared with control animals (Luo et al., 2001 Nature Neuroscience 4:231-232). This evidence further supports the proposal that inhibition of beta-secretase activity and reduction of A beta in the brain provides a therapeutic method for the treatment of AD and other beta amyloid disorders.

At present there are no effective treatments for halting, preventing, or reversing the progression of Alzheimer's disease. Therefore, there is an urgent need for pharmaceutical agents capable of slowing the progression of Alzheimer's disease and/or preventing it in the first place. For example, compounds that are effective inhibitors of beta-secretase, that inhibit beta-secretase-mediated cleavage of APP, that are effective inhibitors of A beta production, and/or are effective to reduce amyloid beta deposits or plaques, are needed for the treatment and prevention of disease characterized by amyloid beta deposits or plaques, such as AD.

### SUMMARY OF THE INVENTION

This invention is directed to compounds of the formula I 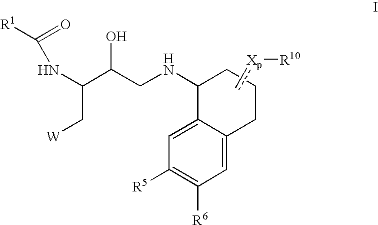
or a pharmaceutically acceptable salt thereof,

wherein

W is selected from the group consisting of (i) C₆-C₁₀ aryl optionally substituted with one to three substituents each independently selected from H, halogen, CN, NO₂, OH, O—C₁-C₈ alkyl, NH₂, NH—C₁-C₈alkyl, N(C₁-C₈alkyl)₂, NHCO—C₁-C₈alkyl, and CONH—C₁-C₈alkyl, and (ii) C₅-C₁₀ heteroaryl optionally substituted with one to three substituents each independently selected from H, halogen, CN, NO₂, OH, O—C₁-C₈ alkyl, NH₂, NH—C₁-C₈ alkyl, N(C₁-C₈ alkyl)₂, NHCO—C₁-C₈alkyl, and CONH—C₁-C₈alkyl,

R¹ is C₁-C₆ alkyl, optionally substituted with up to three fluoro atoms, such as, for example, —CH₂F₂, CHF₂, or —CF₃;

R⁵ and R⁶ are each independently selected from the group consisting of H, C₁-C₈ alkyl, C₂-C₈ alkenyl, C₃-C₈ alkynyl, C₃-C₈ cycloalkyl, 5-10 membered heteroaryl, and C₆-C₁₄ aryl;

p is from 0 to 1,

[symbol] represents a single or double bond, with the proviso that if [symbol] is a double bond, X is N, C(C₁-C₆ alkyl), or O, with the proviso that if X is O and p is 1, R¹⁰ is absent; and with the proviso that if [symbol] is a single bond, X is C₀-C₆ alkylene, C(═C₁-C₆ alkylidene), C(═N—O—C₁-C₆ alkyl), C(═N—C₁-C₆ alkyl), C═O, NH, NC₁-C₆ alkyl, or O; and

R¹⁰ is selected from the group consisting of H, C₁-C₆ alkyl, —OH, —O—C₁-C₆ alkyl, —CO—C₁-C₆ alkyl, —COO—C₁-C₆ alkyl, —NH—C₁-C₆ alkyl, —N(C₁-C₆ alkyl)-C₁-C₆ alkyl, —CO—N(C₁-C₆ alkyl)-C₁-C₆ alkyl, 5-10 membered heteroaryl, 5-10-membered heterocycloalkyl, C₆-C₁₄ aryl, S(O)_{q}C₁-C₆ alkyl, and S(O)_{q}C₆-C₁₀ aryl.

This invention is also directed to:

a pharmaceutical composition for treating, for example, a disorder or condition that may be treated by inhibiting β-secretase, the composition comprising a compound of formula I or a pharmaceutically acceptable salt thereof and a pharmaceutically acceptable carrier;

a method of treatment of a disorder or condition that may be treated by inhibiting β-secretase, the method comprising administering to a mammal in need of such treatment a compound of formula I or a pharmaceutically acceptable salt thereof;

a pharmaceutical composition for treating, for example, a disorder or condition selected from the group consisting of Alzheimer's disease (AD), mild cognitive impairment, Down's syndrome, Hereditary Cerebral Hemorrhage with Amyloidosis of the Dutch-Type, cerebral amyloid angiopathy, dementias of mixed vascular and degenerative origin, dementia associated with Parkinson's disease, dementia associated with progressive supranuclear palsy, dementia associated with cortical basal degeneration, multi-infarct dementia, alcoholic dementia or other drug-related dementia, dementia associated with intracranial tumors or cerebral trauma, dementia associated with Huntington's disease, or AIDS-related dementia, diffuse Lewy body type of Alzheimer's disease, frontotemporal dementias with parkinsonism (FTDP), inclusion body myocytis, supranuclear cataracts, age-related macular degeneration (AMD), Huntington's disease, Parkinson's Disease, Restless Leg Syndrome, stroke, head trauma, spinal cord injury, demyelinating diseases of the nervous system, peripheral neuropathy, pin, cerebral amyloid angiopathy, amyotrophic lateral sclerosis, multiple sclerosis, dyskinesia associated with dopamine agonist therapy, mental retardation, learning disorders, including reading disorder, mathematics disorder, or a disorder of written expression; age-related cognitive decline, amnesic disorders, neuroleptic-induced parkinsonism, tardive dyskinesias, Tourette's syndrome, Multiple Sclerosis, and acute and chronic neurodegenerative disorders, the composition comprising a compound of formula I or a pharmaceutically acceptable salt thereof and a pharmaceutically acceptable carrier;

a method of treatment of a disorder or condition selected from the group consisting of the disorders or conditions listed herein, the method comprising administering to a mammal in need of such treatment a compound of formula I or a pharmaceutically acceptable salt thereof;

a pharmaceutical composition for preventing or delaying the onset of AD, preventing or delaying the onset of AD in patients who would otherwise be expected to progress from mild cognitive impairment (MC1) to AD, or preventing potential consequences of cerebral amyloid angiopathy such as single and recurrent lobar hemorrhages, the composition comprising a compound of formula I or a pharmaceutically acceptable salt thereof and a pharmaceutically acceptable carrier; and

a method for preventing or delaying the onset of AD, preventing or delaying the onset of AD in patients who would otherwise be expected to progress from MCI to AD, or preventing potential consequences of cerebral amyloid angiopathy such as single and recurrent lobar hemorrhages, the method comprising administering to a patient in need of such treatment a compound of formula I or a pharmaceutically acceptable salt thereof.

The invention also provides the use of a compound or salt according to formula I for the manufacture of a medicament.

The invention also provides compounds, pharmaceutical compositions, kits, and methods for inhibiting beta-secretase-mediated cleavage of amyloid precursor protein (APP), protein, the method comprising administering to a patient in need of such treatment a compound of formula I or a pharmaceutically acceptable salt thereof. More particularly, the compounds, compositions, and methods of the invention are effective to inhibit the production of A-beta and to treat or prevent any human or veterinary disease or condition associated with a pathological form of A-beta.

The compounds of the invention possess beta-secretase inhibitory activity. The inhibitory activities of the compounds of the invention is readily demonstrated, for example, using one or more of the assays described herein or known in the art.

The invention also provides methods of preparing the compounds of the invention and the intermediates used in those methods.

The invention also includes a container kit including one or more containers, each container including one or more unit dose of a compound of formula (I), or a pharmaceutically acceptable salt thereof.

The pharmaceutical composition and method of this invention may also be used for preventing a relapse in any of the disorders or conditions described herein. Preventing such relapse is accomplished by administering to a mammal in need of such prevention a compound of formula I or a pharmaceutically acceptable salt thereof and is contemplated as a method of treatment.

The compound of the present invention may have optical centers and therefore may occur in different enantiomeric configurations. Formula I, as depicted above, includes all enantiomers, diastereomers, and other stereoisomers of the compounds depicted in structural formula I, as well as racemic and other mixtures thereof. Individual isomers can be obtained by known methods, such as optical resolution, optically selective reaction, or chromatographic separation in the preparation of the final product or its intermediate.

Isotopically-labeled compounds of formula I or pharmaceutically acceptable salts thereof, including compounds of formula I isotopically-labeled to be detectable by PET or SPECT, are also within the scope of the invention.

Cis and trans isomers of the compound of formula I or a pharmaceutically acceptable salt thereof are also within the scope of the invention.

Tautomers of the compound of formula I or a pharmaceutically acceptable salt thereof are also within the scope of the invention.

When a first group or substituent is substituted by two or more groups or substituents, the invention includes without limitation embodiments in which a combination of such groups or substituents is present.

When a first group or substituent is substituted by two or more groups or substituents, it is understood that the number of such substituents may not exceed the number of positions in the first group or substituent that are available for substitution.

The term “alkyl” refers to straight or branched saturated hydrocarbon radicals. Exemplary alkyl groups include methyl, ethyl, propyl, isopropyl, butyl, isobutyl, pentyl, isopentyl, hexyl, and the like. The term “alkenyl” is used to denote straight or branched hydrocarbon radicals having one or more carbon-carbon double bonds, such as vinyl, allyl, butenyl, and the like. The term “alkynyl” is used to denote straight or branched hydrocarbon radicals having one or more carbon-carbon triple bonds, such as ethynyl, propargyl, 1-butynyl, 2-butynyl, and the like.

By “aryl” is meant an organic radical derived from an aromatic hydrocarbon by removal of a hydrogen from a carbon on an aromatic ring. By “aromatic hydrocarbon” is meant a hydrocarbon having one or more rings in which at least one is aromatic (e.g., phenyl, 1,2,3,4-tetrahydronaphthyl, naphthyl). Preferred aryl groups of the present invention are phenyl, 1-naphthyl, 2-naphthyl, indanyl, indenyl, dihydronaphthyl, fluorenyl, tetralinyl or 6,7,8,9-tetrahydro-5H-benzo[a]cycloheptenyl. The aryl groups herein may be unsubstituted or substituted at one, two, three or four substitutable positions with various substituents. Such substituents include C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

The terms “alkoxy” and “aryloxy” denote “O-alkyl” and “O-aryl”, respectively.

The term “cycloalkyl” denotes a radical formed from a saturated carbocycle by removal of a hydrogen from a ring carbon. Examples of cycloalkyl groups include cyclopropyl, cyclobutyl, cyclopentyl, cyclohexyl, cycloheptyl, and the like. As used herein, the term “cycloalkyl” is also intended to denote a radical formed from a saturated carbocycle comprising at least two fused rings, such as adamantanyl, decahydronaphthalinyl, norbornanyl, and the like.

The term “halo” represents chloro, fluoro, bromo, and iodo.

The term “heteroaryl” denotes an organic radical derived from a heteroaromatic hydrocarbon by removal of a hydrogen from a carbon on an aromatic ring, wherein a “heteroaromatic hydrocarbon” is an aromatic hydrocarbon containing one or more ring heteroatoms selected from the group consisting of nitrogen, oxygen, and sulfur. If the heteroaryl group contains more than one heteroatom, the heteroatoms may be the same or different. Preferred heteroaryl groups include five- and six-membered rings that contain from one to four heteroatoms independently selected from oxygen, nitrogen, and sulfur. Examples of preferred five- and six-membered heteroaryl groups include benzo[b]thienyl, chromenyl, furyl, imidazolyl, indazolyl, indolizinyl, indolyl, isobenzofuranyl, isoindolyl, isoquinolyl, isothiazolyl, isoxazolyl, naphthyridinyl, oxadiazolyl, oxazinyl, oxazolyl, phthalazinyl, pteridinyl, purinyl, pyranyl, pyrazinyl, pyrazolyl, pyridazinyl, pyridyl, pyrimidyl, pyrrolyl, quinolizinyl, quinolyl, quinoxalinyl, thiazolyl, thienyl, triazinyl, triazolyl, tetrazolyl and xanthenyl. Preferred heteroaryl groups of the present invention also include indolinyl, quinazolinyl, benzothiazolyl, benzimidazolyl, benzofuranyl, furanyl, thienyl, thiadiazolyl, oxazolopyridinyl, imidazopyridinyl, naphthyridinyl, cinnolinyl, carbazolyl, beta-carbolinyl, isochromanyl, chromanyl, tetrahydroisoquinolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, benzodioxolyl, phenoxazinyl, phenothiazinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide, tetrahydrocarbazole, tetrahydrobetacarboline. The heteroaryl groups herein are unsubstituted or, as specified, substituted in one or more substitutable positions with various groups. For example, such heteroaryl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

The term “heterocycloalkyl” denotes a cycloalkyl system, wherein “cycloalkyl” is defined above, in which one or more of the ring carbon atoms are replaced with a heteroatom selected from the group consisting of nitrogen, oxygen, and sulfur. If the heterocycloalkyl group contains more than one heteroatom, the heteroatoms may be the same or different. Examples of such heterocycloalkyl groups include azabicycloheptanyl, azetidinyl, benzazepinyl, 1,3-dihydroisoindolyl, indolinyl, tetrahydrofuryl, tetrahydroquinolinyl, tetrahydroisoquinolinyl, morpholinyl, piperazinyl, piperidyl, pyrrolidinyl, and, tetrahydro-2H-1,4-thiazinyl.

The term “alkylene” refers to methylene or to straight or branched saturated hydrocarbon diradicals having more than one carbon in which each of two different carbons has a free valence. Exemplary alkylene groups include methylene, —CH₂CH₂—, —CH₂CH₂CH₂—, —CH₂CH(CH₃)—, —CH₂CH₂CH₂CH₂—, —CH₂CH(CH₃)CH₂—, —CH₂CH₂CH₂CH₂CH₂—, —CH₂CH(CH₃)CH₂CH₂—, —CH₂CH₂CH₂CH₂CH₂CH₂—, and the like.

The term “alkylidene” refers to straight or branched saturated hydrocarbon diradicals, in which a carbon has two free valences. Exemplary alkylidene groups include methylene, CH(CH₃), C(CH₃)₂, C(CH₃)C₂H₅, and the like.

A cyclic group may be bonded to another group in more than one way. If no particular bonding arrangement is specified, then all possible arrangements are intended. For example, the term “pyridyl” includes 2-, 3-, or 4-pyridyl, and the term “thienyl” includes 2- or 3-thienyl.

As used herein, “mammal” is any member of the class mammalia. As an example, the mammal, in need of the treatment or prevention may be a human. As another example, the mammal in need of the treatment or prevention may be a mammal other than a human.

APP, amyloid precursor protein, is defined as any APP polypeptide, including APP variants, mutations, and isoforms, for example, as disclosed in U.S. Pat. No. 5,766,846.

A beta, amyloid beta peptide, is defined as any peptide resulting from beta-secretase mediated cleavage of APP, including peptides of 39, 40, 41, 42, and 43 amino acids, and extending from the beta-secretase cleavage site to amino acids 39, 40, 41, 42, or 43.

Beta-secretase (BACE1, Asp2, Memapsin 2) is an aspartyl protease that mediates cleavage of APP at the amino-terminal edge of A beta. Human beta-secretase is described, for example, in WO00/17369.

Preferably, the compositions and methods of the invention employ a therapeutically effective amount of the compound of formula I.

A compound of formula I which is basic in nature is capable of forming a wide variety of different salts with various inorganic and organic acids. The acid addition salts are readily prepared by treating the base compounds with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid salts of the active compound used in formulating the pharmaceutical composition of this invention that are basic in nature are those which form non-toxic acid addition salts, i.e., salts containing pharmacologically acceptable anions. Non-limiting examples of the salts include the acetate, benzoate, beta.-hydroxybutyrate, bisulfate, bisulfite, bromide, butyne-1,4-dioate, carpoate, chloride, chlorobenzoate, citrate, dihydrogenphosphate, dinitrobenzoate, fumarate, glycolate, heptanoate, hexyne-1,6-dioate, hydroxybenzoate, iodide, lactate, maleate, malonate, mandelate, metaphosphate, methanesulfonate, methoxybenzoate, methylbenzoate, monohydrogenphosphate, naphthalene-1-sulfonate, naphthalene-2-sulfonate, oxalate, phenylbutyrate, phenylproionate, phosphate, phthalate, phenylacetate, propanesulfonate, propiolate, propionate, pyrophosphate, pyrosulfate, sebacate, suberate, succinate, sulfate, sulfite, sulfonate, tartrate, xylenesulfonate, acid phosphate, acid citrate, bitartrate, succinate, gluconate, saccharate, nitrate, methanesulfonate, pamoate [i.e., 1,1′-methylene-bis-(2-hydroxy-3-naphthoate)] salts, and salts of the following acids: CH₃—(CH₂)ₙ—COOH where n is 0 thru 4, and HOOC—(CH₂)ₙ—COOH where n is as defined above.

In an exemplary embodiment, the compound of formula I is a compound of formula 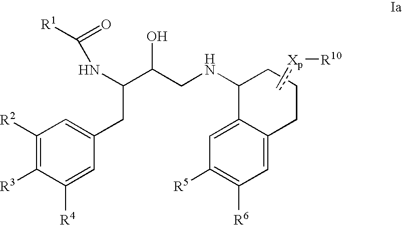
wherein R², R³, R⁴ are each independently selected from the group consisting of H, halogen, CN, NO₂, OH, O—C₁-C₈ alkyl, NH₂, NH—C₁-C₈ alkyl, N(C₁-C₈ alkyl)₂, NHCO—C₁-C₈ alkyl, and CONH—C₁-C₈.

Exemplary embodiments of the present invention include the compound of formula Ia in which:

(A) R² is fluoro;

(B) R⁴ is fluoro;

(C) R³ is H;

Exemplary embodiments of the present invention include the compound of formula I or Ia in which:

(D) R¹ is CH₃;

(E) R⁵ is neopentyl or t-butyl;

(F) R⁶ is H;

(G) [symbol] represents a single bond and X is C(═C₁-C₆ alkylidene), C(═N—O—C₁-C₆ alkyl), C(═N—C₁-C₆ alkyl), C═O, NH, NC₁-C₆ alkyl, or O;

(H) [symbol] represents a double bond and X is N or C(C₁-C₆ alkyl); and

(I) R¹⁰ is selected from the group consisting of H, C₁-C₆ alkyl, —OH, —O—C₁-C₆ alkyl, —CO—C₁-C₆ alkyl, —COO—C₁-C₆ alkyl, —NH—C₁-C₆ alkyl, —CO—N(C₁-C₆ alkyl)-C₁-C₆ alkyl, 5-10 membered heteroaryl, 5-10-membered heterocycloalkyl, and C₆-C₁₄ aryl.

Exemplary embodiments of the present invention also include any combination of the foregoing embodiments (A)-(I).

Exemplary embodiments of the present invention also include the following optical isomers of compounds of the formula Ia and their enantiomers:

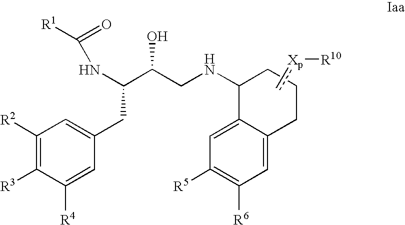

The compound of formula I may be prepared by a process comprising

(i) reacting a compound of formula V 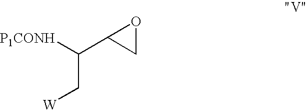
wherein P₁ is R¹ or R¹—O, with a compound of formula IV

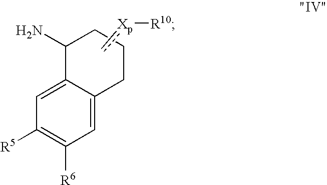

(ii) removing the P₁CO group from the product formed in (i); and

(iii) reacting the product formed in (ii) with a compound capable of transferring an R¹CO group, to form I,

with the proviso that, if P₁═R¹, steps (ii) and (iii) are absent.

As used herein, “removing the P₁CO group from the product formed in (i)” refers to performing a deprotection step in any manner that is known to a person skilled in the art. Such a step may be, but is not limited to, a hydrolysis of the P₁CO—N bond, or a reaction of the product formed in (i) with a nucleophile capable of performing a nucleophilic substitution at the acyl carbon of the P₁CO—NH. For example, acid or basic hydrolysis may be used to hydrolyze the P₁CO—N bond, thereby removing the PICO group.

As used herein, “a compound capable of transferring an R¹CO group” refers to any compound of the formula R₁COX², wherein X² is a leaving group. For example, X² may be, but is not limited to, a halogen, such as, for example, chloride, or a cyano group, or a C₁-C₆ alkoxy group, or a C₁-C₆ alkylthio group.

Alternatively, the compounds of formula I may be prepared, for example, by a process comprising

(i) reacting a compound of the formula II 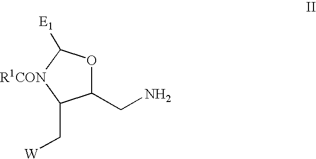
wherein E₁ is (C₁-C₁₂ alkyl) or (C₆-C₁₀ aryl) optionally substituted with C₁-C₆ alkyl, C₁-C₆ alkoxy, or halo, with a compound of the formula III
and

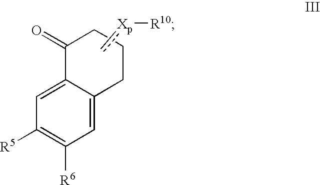

(ii) treating the product formed in (i) with a reducing agent containing a boron-hydrogen bond. The reducing agent may be, for example, a borohydride.

The process may further comprise

(iii) treating the product formed in (ii) with acid in water, a C₁-C₄ alkyl alcohol, or a mixture of water and a C₁-C₄ alkyl alcohol.

The compound of formula II may be, for example, a compound having of the formula IIa:

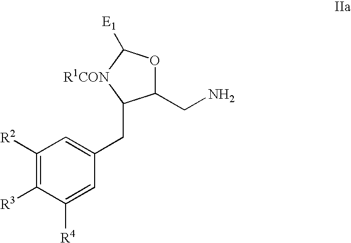

As an example, shown in Scheme 1, substituted aminotetralin 4 can be reacted with epoxide 5 in a suitably inert solvent such as THF, dioxane or alcohols where isopropanol is preferred at a temperature range of 50 to 150° C. where 80-120° C. is preferred to afford the BOC protected intermediate 3. Deprotection of 3 can be accomplished by treatment with acids such as trifluoroacetic acid, aqueous sulfuric acid or preferably aqueous hydrochloric acid to afford 2. Compound 1 can be prepared by acetylation of 2 using acetyl chloride and tertiary amine base, acetyl imidazole or preferably with acetic acid and an amide coupling agent such as EDC.HCl.

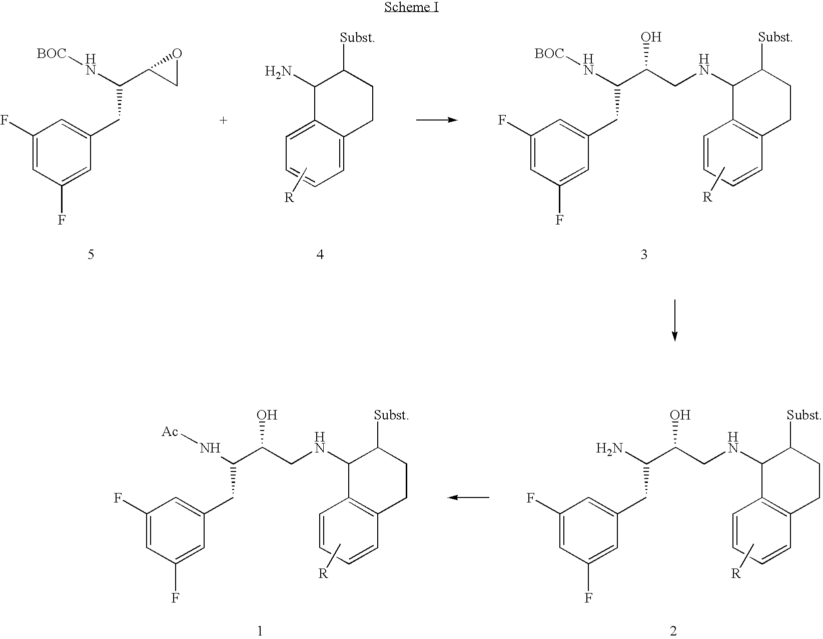

As another example, shown in Scheme 2, substituted tetralones 6a-i can be submitted to reductive amination conditions with the protected amine 7 using NaBCNH₃ in acetic acid or preferably NaB(OAc)₃H in 1,2-dichloroethane and then treated with aqueous acid in methanol to afford the compound of formula I.

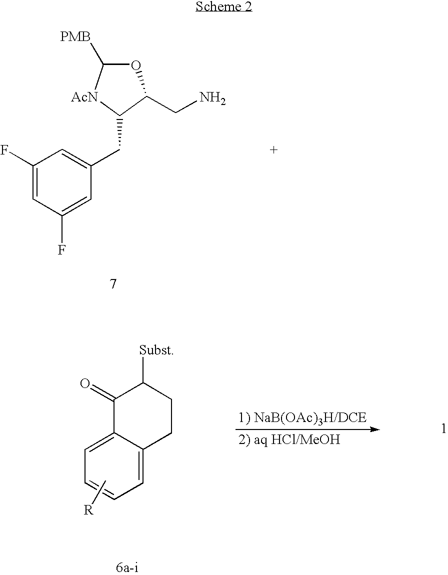

One skilled in the art will readily appreciate that a variety of substitutions and other modifications can be made to the structures of the reactants in each step of either Scheme 1 or Scheme 2 without affecting the successful outcome of each step or of the entire Scheme.

Preparation of examples of the intermediate tetralones 6a-h and of examples of the intermediate amino-tetralines 4 is shown in Scheme 3. Halogenated alpha-tetralone 61 can be converted to alkylated tetralones of type 6h via Negishi type coupling with alkyl zinc reagents in the presence of palladium catalysts preferably Pd(dppf)Cl₂ in inert solvents such as diethylether or preferably THF. 6h may be converted into other tetralone intermediates. Thus treatment of 6h with aryl halides following the procedure described by Nolan et al. (Org. Lett., 2002, 4, 4053) can provide aryl or heteroaryl substituted tetralone 6c. Alpha halogenation of 6h with CuBr₂ in refluxing ethyl acetate or bromine in unreactive solvents such as acetic acid or diethylether gives 6g which may then be treated with various nucleophiles such as, but not limited to, amines, alkoxides, sulfides, to afford compounds of formula 6a. Compounds of formula 6c may also be obtained from treatment of 6g with aryl Grignard reagents following the procedure described by Hussey and Herr (JOC, 1959, 24, 843). Condensation of 6h with various aryl, heteroaryl and alkyl aldehydes following procedures such as those described by Wachter, et al (J. Med. Chem., 1996, 39, 834) can provide olefins 6f which may then be converted to compound 6e by treatment with a reducing agent such as hydrogen with a metal catalyst such as palladium on carbon. Oxime 6b can be prepared from 6h according to the condensation procedure of Oka, et al. (Chem.Pharm.Bull., 1977, 25, 632). Ketoester compounds of formula 6d can be prepared by treatment of 6h with bases such as sodium hydride or sodium ethoxide followed by reaction with dialkylcarbonates according to the general procedures described by Chakrabarty, et al. (Synthesis, 2003, 15, 2294). All compounds of type 6a-i may be further converted into other species using methods that will be obvious to one skilled in the art.

Alpha amine tetralins of formula 4 can be prepared from tetralones of formula 6a-i. Treatment of 6a-i with hydroxylamine hydrochloride in the presence of a base such as sodium ethoxide or pyridine will afford the oxime which can then be reduced with metal hydrides such as lithium aluminum hydride, or borane or hydrogenated in the presence of Raney nickel or palladium on carbon to form amino-tetralin 4. Likewise, reductive amination of 6a-i with, for instance, ammonium acetate and a boron hydride such as sodium borohydride, sodium cyano borohydride or sodium triacetoxyborohydride can also give compound 4.

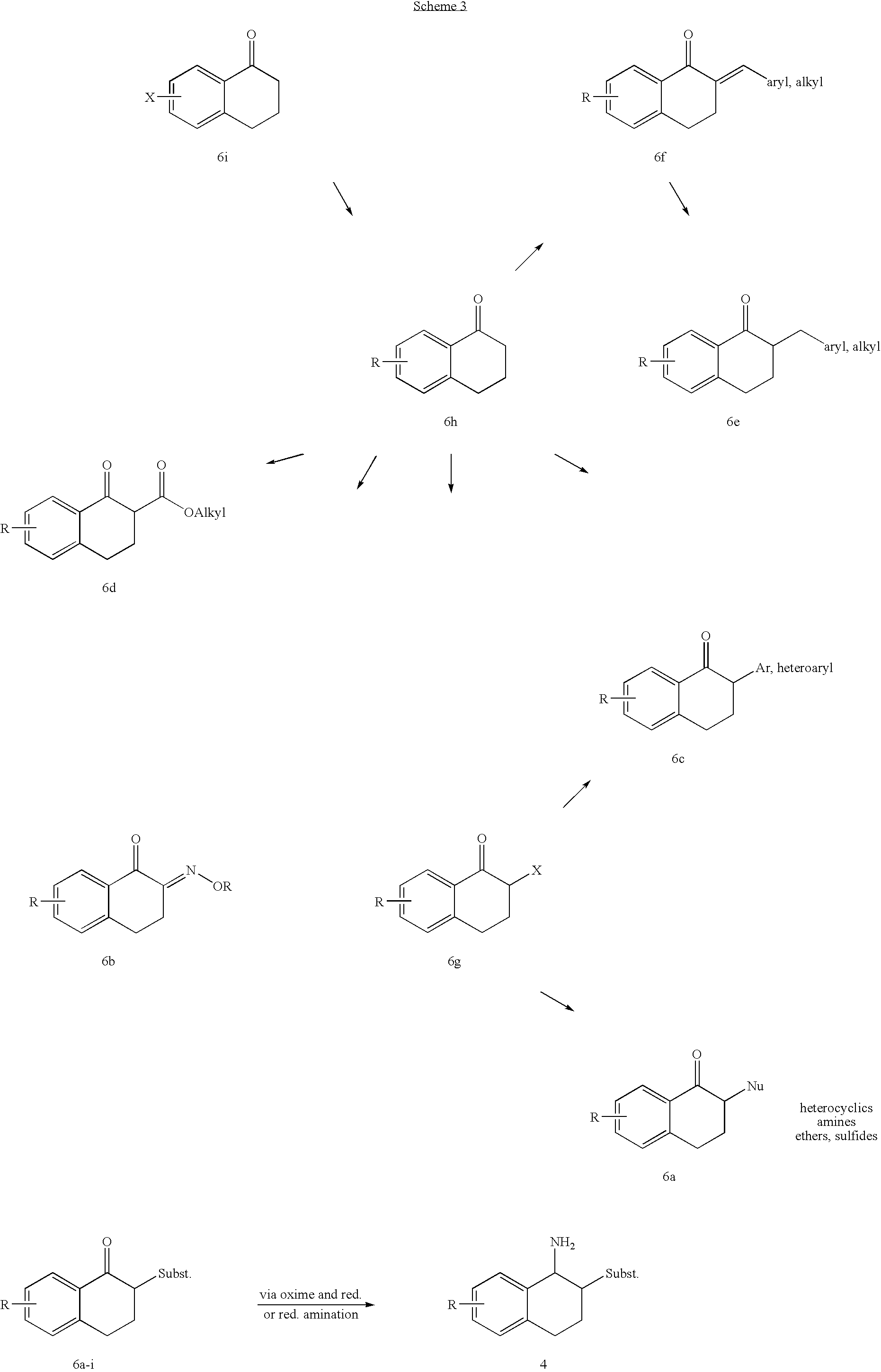

### DETAILED DESCRIPTION OF THE INVENTION

Exemplary compounds of the invention include the ones shown below:
N-{1-(3,5-Difluoro-benzyl)-3-[7-(2,2-dimethyl-propyl)-2-piperidin-1-yl-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-phenyl-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-(1H-imidazol-2-yl)-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-morpholin-4-yl-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-methylamino-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-methoxy-1,2,3,4-tetrahydro-naphthalen-1-ylamino]-2-hydroxy-propyl}-acetamide;
1-[3-Acetylamino-4-(3,5-difluoro-phenyl)-2-hydroxy-butylamino]-7-(2,2-dimethyl-propyl)-1,2,3,4-tetrahydro-naphthalene-2-carboxylic acid methyl ester;
1-[3-Acetylamino-4-(3,5-difluoro-phenyl)-2-hydroxy-butylamino]-7-(2,2-dimethyl-propyl)-1,2,3,4-tetrahydro-naphthalene-2-carboxylic acid dimethylamide;
N-[3-[2-Acetyl-7-(2,2-dimethyl-propyl)-1,2,3,4-tetrahydro-naphthalen-1-ylamino]-1-(3,5-difluoro-benzyl)-2-hydroxy-propyl]-acetamide;
Dimethyl-carbamic acid 1-[3-acetylamino-4-(3,5-difluoro-phenyl)-2-hydroxy-butylamino]-7-(2,2-dimethyl-propyl)-1,2,3,4-tetrahydro-naphthalen-2-yl ester;
Carbonic acid 1-[3-acetylamino-4-(3,5-difluoro-phenyl)-2-hydroxy-butylamino]-7-(2,2-dimethyl-propyl)-1,2,3,4-tetrahydro-naphthalen-2-yl ester methyl ester;
N-{1-(3,5-Difluoro-benzyl)-3-[7-(2,2-dimethyl-propyl)-2-(1,3,3-trimethyl-ureido)-1,2,3,4-tetrahydro-naphthalen-1-ylamino]-2-hydroxy-propyl}-acetamide;
[1-[3-Acetylamino-4-(3,5-difluoro-phenyl)-2-hydroxy-butylamino]-7-(2,2-dimethyl-propyl)-1,2,3,4-tetrahydro-naphthalen-2-yl]-methyl-carbamic acid methyl ester;
N-{1-(3,5-Difluoro-benzyl)-3-[7-(2,2-dimethyl-propyl)-2-(1-methoxyimino-ethyl)-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-(1-methoxy-ethyl)-1,2,3,4-tetrahydro-naphthalen-1-ylamino]-2-hydroxy-propyl}-acetamide;
N-[3-[2-Acetylamino-7-(2,2-dimethyl-propyl)-1,2,3,4-tetrahydro-naphthalen-1-ylamino]-1-(3,5-difluoro-benzyl)-2-hydroxy-propyl]-acetamide;
N-{1-(3,5-Difluoro-benzyl)-3-[7-(2,2-dimethyl-propyl)-2-methanesulfonylamino-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-(3-methyl-3H-imidazol-4-ylmethylene)-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-sulfamoyl-1,2,3,4-tetrahydro-naphthalen-1-ylamino]-2-hydroxy-propyl}-acetamide;
N-[3-[7-(2,2-Dimethyl-propyl)-2-morpholin-4-yl-1,2,3,4-tetrahydro-naphthalen-1-ylamino]-1-(4-fluoro-pyridin-2-ylmethyl)-2-hydroxy-propyl]-acetamide; and
1-[3-Acetylamino-4-(2,6-difluoro-pyrimidin-4-yl)-2-hydroxy-butylamino]-7-(2,2-dimethyl-propyl)-1,2,3,4-tetrahydro-naphthalene-2-carboxylic acid dimethylamide.

The pharmaceutical composition of the present invention may be a composition comprising a compound of formula I and a pharmaceutically acceptable carrier. The pharmaceutical composition may be formulated in a conventional manner using one or more pharmaceutically acceptable carriers. The composition may be formulated for oral, buccal, intranasal, parenteral (e.g., intravenous, intramuscular, intraperitoneal, or subcutaneous or through an implant) nasal, vaginal, sublingual, rectal or topical administration or in a form suitable for administration by inhalation or insufflation. In the composition of the invention, the compound of formula I is preferably present in an amount effective in treating a condition as described herein. Similarly, in the method of treatment of a condition as described herein, the amount of compound of formula I administered is preferably an amount effective in treating the condition.

The invention includes a method for treating a patient who has, or for preventing a patient from getting, a disease or condition described herein.

In an embodiment, the method of treatment of the invention is used to treat Alzheimer's disease.

In an embodiment, the method of treatment can help prevent or delay the onset of Alzheimer's disease.

In an embodiment, the method of treatment can be used where the disease is mild cognitive impairment.

In an embodiment, the method of treatment can be used where the disease is Down's syndrome.

In an embodiment, the method of treatment can be used where the disease is Hereditary Cerebral Hemorrhage with Amyloidosis of the Dutch-Type.

In an embodiment, the method of treatment can be used where the disease is cerebral amyloid angiopathy.

In an embodiment, the method of treatment can be used where the disease is a degenerative dementia or dementias.

In an embodiment, the method of treatment can be used where the disease is diffuse Lewy body type of Alzheimer's disease.

In an embodiment, the method of treatment can treat an existing disease.

In an embodiment, the method of treatment can prevent a disease from developing.

In an embodiment, the method of treatment can employ therapeutically effective amounts: for oral administration from about 0.1 mg/day to about 1,000 mg/day; for parenteral, sublingual, intranasal, intrathecal administration from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg.

In an embodiment, this method of treatment can employ therapeutically effective amounts: for oral administration from about 1 mg/day to about 100 mg/day; and for parenteral administration from about 5 to about 50 mg daily.

In an embodiment, this method of treatment can employ therapeutically effective amounts for oral administration from about 5 mg/day to about 50 mg/day.

The invention also includes pharmaceutical compositions which include a compound of formula (I) or a pharmaceutically acceptable salts thereof.

The invention includes the use of a compound of formula (I) or pharmaceutically acceptable salts thereof for the manufacture of a medicament for use in treating a patient who has, or in preventing a patient from getting, a disease or condition described herein.

In an embodiment, this use of a compound of formula (I) can be employed where the disease is Alzheimer's disease.

In an embodiment, this use of a compound of formula (I) can help prevent or delay the onset of Alzheimer's disease.

In an embodiment, this use of a compound of formula (I) can be employed where the disease is mild cognitive impairment.

In an embodiment, this use of a compound of formula (I) can be employed where the disease is Down's syndrome.

In an embodiment, this use of a compound of formula (I) can be employed where the disease is Hereditary Cerebral Hemorrhage with Amyloidosis of the Dutch-Type.

In an embodiment, this use of a compound of formula (I) can be employed where the disease is cerebral amyloid angiopathy.

In an embodiment, this use of a compound of formula (I) can be employed where the disease is a degenerative dementia or dementias.

In an embodiment, this use of a compound of formula (I) can be employed where the disease is diffuse Lewy body type of Alzheimer's disease.

In an embodiment, this use of a compound employs a pharmaceutically acceptable salt selected from the group consisting of salts of the following acids hydrochloric, hydrobromic, hydroiodic, nitric, sulfuric, phosphoric, citric, methanesulfonic, CH₃—(CH₂)ₙ—COOH where n is 0 thru 4, HOOC—(CH₂)n—COOH where n is as defined above, HOOC—CH═CH—COOH, and phenyl-COOH, acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic and toluenesulfonic. For other acceptable salts, see Int. J. Pharm., 33, 201-217 (1986) and J. Pharm. Sci., 66(1), 1, (1977).

These methods each include administration of a therapeutically effective amount of a compound of formula (I) or a pharmaceutically acceptable salts thereof.

The invention also includes a method for inhibiting beta-secretase activity, including exposing said beta-secretase to an effective inhibitory amount of a compound of formula (I), or a pharmaceutically acceptable salt thereof.

In an embodiment, this method employs a compound that inhibits 50% of the enzyme's activity at a concentration of less than 50 micromolar.

In an embodiment, this method employs a compound that inhibits 50% of the enzyme's activity at a concentration of 10 micromolar or less.

In an embodiment, this method employs a compound that inhibits 50% of the enzyme's activity at a concentration of 1 micromolar or less.

In an embodiment, this method employs a compound that inhibits 50% of the enzyme's activity at a concentration of less than 100 nanomolar.

In an embodiment, this method employs a compound that inhibits 50% of the enzyme's activity at a concentration of 10 nanomolar or less.

In an embodiment, this method includes exposing said beta-secretase to said compound in vitro.

In an embodiment, this method includes exposing said beta-secretase to said compound in a cell.

In an embodiment, this method includes exposing said beta-secretase to said compound in a cell in an animal.

In an embodiment, this method includes exposing said beta-secretase to said compound in a human.

The invention also includes methods for inhibiting beta-secretase activity, for inhibiting cleavage of amyloid precursor protein (APP), in a reaction mixture, at a site between Met596 and Asp597, numbered for the APP-695 amino acid isotype, or at a corresponding site of an isotype or mutant thereof; for inhibiting production of amyloid beta peptide (A beta) in a cell; for inhibiting the production of beta-amyloid plaque in an animal; and for treating or preventing a disease characterized by beta-amyloid deposits in the brain. including exposing said reaction mixture to an effective inhibitory amount of a compound of formula (I), or a pharmaceutically acceptable salt thereof.

In an embodiment, this method employs a cleavage site: between Met652 and Asp653, numbered for the APP-751 isotype; between Met 671 and Asp 672, numbered for the APP-770 isotype; between Leu596 and Asp597 of the APP-695 Swedish Mutation; between Leu652 and Asp653 of the APP-751 Swedish Mutation; or between Leu671 and Asp672 of the APP-770 Swedish Mutation.

In an embodiment, this method exposes said reaction mixture in vitro.

In an embodiment, this method exposes said reaction mixture in a cell.

In an embodiment, this method exposes said reaction mixture in an animal cell.

In an embodiment, this method exposes said reaction mixture in a human cell.

The invention also includes a method for inhibiting production of amyloid beta peptide (A beta) in a cell, including administering to said cell an effective inhibitory amount of a compound of formula (I), or a pharmaceutically acceptable salt thereof.

In an embodiment, this method includes administering to an animal.

In an embodiment, this method includes administering to a human.

The invention also includes a method for inhibiting the production of beta-amyloid plaque in an animal, including administering to said animal an effective inhibitory amount of a compound of formula (I), or a pharmaceutically acceptable salt thereof.

In an embodiment, this method includes administering to a human.

The invention also includes a method for treating or preventing a disease characterized by beta-amyloid deposits in the brain including administering to a patient an effective therapeutic amount of a compound of formula (I), or a pharmaceutically acceptable salt thereof.

In an embodiment, this method employs a compound that inhibits 50% of the enzyme's activity at a concentration of less than 50 micromolar.

In an embodiment, this method employs a compound that inhibits 50% of the enzyme's activity at a concentration of 10 micromolar or less.

In an embodiment, this method employs a compound that inhibits 50% of the enzyme's activity at a concentration of 1 micromolar or less.

In an embodiment, this method employs a compound that inhibits 50% of the enzyme's activity at a concentration of 10 nanomolar or less.

In an embodiment, this method employs a compound at a therapeutic amount in the range of from about 0.1 to about 1000 mg/day.

In an embodiment, this method employs a compound at a therapeutic amount in the range of from about 15 to about 1500 mg/day.

In an embodiment, this method employs a compound at a therapeutic amount in the range of from about 1 to about 100 mg/day.

In an embodiment, this method employs a compound at a therapeutic amount in the range of from about 5 to about 50 mg/day.

The invention also includes a method for producing a beta-secretase complex including exposing beta-secretase to a compound of formula (I), or a pharmaceutically acceptable salt thereof, in a reaction mixture under conditions suitable for the production of said complex.

In an embodiment, this method employs exposing in vitro.

In an embodiment, this method employs a reaction mixture that is a cell.

The invention also includes a component kit including component parts capable of being assembled, in which at least one component part includes a compound of formula I enclosed in a container.

In an embodiment, this component kit includes lyophilized compound, and at least one further component part includes a diluent.

The invention also includes a container kit including one or more containers, each container including one or more unit dose of a compound of formula (I), or a pharmaceutically acceptable salt thereof.

In an embodiment, this container kit includes each container adapted for oral delivery and includes a tablet, gel, or capsule.

In an embodiment, this container kit includes each container adapted for parenteral delivery and includes a depot product, syringe, ampoule, or vial.

In an embodiment, this container kit includes each container adapted for topical delivery and includes a patch, medipad, ointment, or cream.

The invention also includes an agent kit including a compound of formula (I), or a pharmaceutically acceptable salt thereof; and one or more therapeutic agent selected from the group consisting of an antioxidant, an anti-inflammatory, a gamma secretase inhibitor, a neurotrophic agent, an acetyl cholinesterase inhibitor, a statin, A beta, and an anti-A beta antibody.

The invention also includes a composition including a compound of formula (I), or a pharmaceutically acceptable salt thereof; and an inert diluent or edible carrier.

In an embodiment, this composition includes a carrier that is an oil.

The invention also includes a composition including: a compound of formula (I), or a pharmaceutically acceptable salt thereof; and a binder, excipient, disintegrating agent, lubricant, or gildant.

The invention also includes a composition including a compound of formula (I), or a pharmaceutically acceptable salt thereof; disposed in a cream, ointment, or patch.

The invention provides compounds of formula (I), and the other formulas contained herein, that are useful in treating and preventing Alzheimer's disease. In addition to the process shown in Scheme I, examples of methods of preparation in the art that may be applied for the preparation of the compounds of the invention are found in: J. Org. Chem. 1998, 63, 4898-4906; J. Org. Chem. 1997, 62, 9348-9353; J. Org. Chem. 1996, 61, 5528-5531; J. Med. Chem. 1993, 36, 320-330; J. Am. Chem. Soc. 1999, 121, 1145-1155; and references cited therein. See also U.S. Pat. Nos. 6,150,530, 5,892,052, 5,696,270, and 5,362,912 and references cited therein.

The compounds of the invention may contain geometric or optical isomers as tautomers. Thus, the invention includes all tautomers and pure geometric isomers, such as the E and Z geometric isomers, as mixtures thereof. Further, the invention includes pure enantiomers and diastereomers as mixtures thereof, including racemic mixtures. The individual geometric isomers, enantiomers or diastereomers may be prepared or isolated by methods known to those skilled in the art, including but not limited to chiral chromatography; preparing diastereomers, separating the diastereomers and then converting the diastereomers into enantiomers through methods well known in the art.

Compounds of the invention with designated stereochemistry can be included in mixtures, including racemic mixtures, with other enantiomers, diastereomers, geometric isomers or tautomers. In a preferred aspect, compounds of the invention are typically present in these mixtures in diastereomeric and/or enantiomeric excess of at least 50 percent. Preferably, compounds of the invention are present in these mixtures in diastereomeric and/or enantiomeric excess of at least 80 percent. More preferably, compounds of the invention with the desired stereochemistry are present in diastereomeric and/or enantiomeric excess of at least 90 percent. Even more preferably, compounds of the invention with the desired stereochemistry are present in diastereomeric and/or enantiomeric excess of at least 99 percent.

The compound of the invention may be used to treat or prevent the diseases or conditions disclosed herein. When treating or preventing these diseases, the compounds of the invention can either be used individually or in combination, as is best for the patient.

The term “preventing” refers to slowing the development of disease symptoms, delaying the onset of the disease, or prevent a patient from developing a disease at all, where the patient has not been diagnosed as having the disease at the time of administration, but would normally be expected to develop the disease or be at increased risk for the disease. Preventing also includes administration of the compounds of the invention to those individuals thought to be predisposed to the disease due to age, familial history, genetic or chromosomal abnormalities, and/or due to the presence of one or more biological markers for the disease, such as a known genetic mutation of APP or APP cleavage products in brain tissues or fluids.

In treating or preventing the above diseases, the compounds of the invention are preferably administered in a therapeutically effective amount. The therapeutically effective amount will vary depending on the particular compound used and the route of administration, as is known to those skilled in the art.

In treating a patient displaying any of the diagnosed above conditions a physician may administer a compound of the invention immediately and continue administration indefinitely, as needed. In treating patients who are not diagnosed as having Alzheimer's disease, but who are believed to be at substantial risk for Alzheimer's disease, the physician should preferably start treatment when the patient first experiences early pre-Alzheimer's symptoms such as, memory or cognitive problems associated with aging. In addition, there are some patients who may be determined to be at risk for developing Alzheimer's through the detection of a genetic marker such as APOE4 or other biological indicators that are predictive for Alzheimer's disease. In these situations, even though the patient does not have symptoms of the disease, administration of the compounds of the invention may be started before symptoms appear, and treatment may be continued indefinitely to prevent or delay the onset of the disease.

The compounds of the invention can be administered orally, parenterally, (IV, IM, depo-IM, SQ, and depo SQ), sublingually, intranasally (inhalation), intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

About 1 to 500 mg of a compound or mixture of compounds of the invention or a physiologically acceptable salt is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the compounds of the invention.

When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the patient 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit beta-secretase activity, to inhibit A beta production, to inhibit A beta deposition, or to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a patient may be started at one dose, that dose may be varied over time as the patient's condition changes.

Compounds of the invention may also be advantageously delivered in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

The compounds of the invention can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the patients with Alzheimer's disease, it is preferred that the parenteral dosage form be a depo formulation.

The compounds of the invention can be administered sublingually. When given sublingually, the compounds of the invention should be given one to four times daily in the amounts described above for IM administration.

The compounds of the invention can be administered intranasally. When given by this route, the appropriate dosage forms are a nasal spray or dry powder, as is known to those skilled in the art. The dosage of the compounds of the invention for intranasal administration is the amount described above for IM administration.

The compounds of the invention can be administered intrathecally. When given by this route the appropriate dosage form can be a parenteral dosage form as is known to those skilled in the art. The dosage of the compounds of the invention for intrathecal administration is the amount described above for IM administration.

The compounds of the invention can be administered topically. When given by this route, the appropriate dosage form is a cream, ointment, or patch. Because of the amount of the compounds of the invention to be administered, the patch is preferred. When administered topically, the dosage is from about 0.5 mg/day to about 200 mg/day. Because the amount that can be delivered by a patch is limited, two or more patches may be used. The number and size of the patch is not important, what is important is that a therapeutically effective amount of the compounds of the invention be delivered as is known to those skilled in the art. The compounds of the invention can be administered rectally by suppository as is known to those skilled in the art. When administered by suppository, the therapeutically effective amount is from about 0.5 mg to about 500 mg.

Generally, dosage levels of between about 0.01 and about 100 mg/kg of body weight daily are administered to humans and other mammals. A preferred dosage range in humans is about 0.1 to about 50 mg/kg of body weight daily which can be administered as a single dose or divided into multiple doses. A preferred dosage range in mammals other than humans is about 0.01 to about 10.0 mg/kg of body weight daily which can be administered as a single dose or divided into multiple doses. A more preferred dosage range in mammals other than humans is about 0.1 to about 5.0 mg/kg of body weight daily which can be administered as a single dose or divided into multiple doses.

Aerosol formulations for treatment of the conditions referred to herein in the average adult human are preferably arranged so that each metered dose or “puff” of aerosol contains about 20 μg to about 1000 μg of the compound of formula I. The overall daily dose with an aerosol will be within the range about 100 μg to about 10 mg. Administration may be several times daily, for example 2, 3, 4 or 8 times, giving for example, 1, 2 or 3 doses each time.

The compounds of the invention can be administered by implants as is known to those skilled in the art. When administering a compound of the invention by implant, the therapeutically effective amount is the amount described above for depot administration.

Given a particular compound of the invention and a desired dosage form, one skilled in the art would know how to prepare and administer the appropriate dosage form.

The compounds of the invention are used in the same manner, by the same routes of administration, using the same pharmaceutical dosage forms, and at the same dosing schedule as described above, for preventing or treating the diseases or conditions disclosed herein.

The compounds of the invention can be used in combination, with each other or with other therapeutic agents or approaches used to treat or prevent the conditions listed above. Such agents or approaches include: acetylcholine esterase inhibitors such as tacrine (tetrahydroaminoacridine, marketed as COGNEX®), donepezil hydrochloride, (marketed as Aricept® and rivastigmine (marketed as Exelon®); gamma-secretase inhibitors; anti-inflammatory agents such as cyclooxygenase II inhibitors; anti-oxidants such as Vitamin E and ginkolides; immunological approaches, such as, for example, immunization with A beta peptide or administration of anti-A beta peptide antibodies; statins; and direct or indirect neurotropic agents such as Cerebrolysin®, AIT-082 (Emilieu, 2000, Arch. Neurol. 57:454), and other neurotropic agents of the future.

In addition, the compounds of formula (I) can also be used with inhibitors of P-glycoprotein (P-gp). P-gp inhibitors and the use of such compounds are known to those skilled in the art. See for example, Cancer Research, 53, 4595-4602 (1993), Clin. Cancer Res., 2, 7-12 (1996), Cancer Research, 56, 4171-4179 (1996), International Publications WO99/64001 and WO01/10387. The important thing is that the blood level of the P-gp inhibitor be such that it exerts its effect in inhibiting P-gp from decreasing brain blood levels of the compounds of formula (A). To that end the P-gp inhibitor and the compounds of formula (A) can be administered at the same time, by the same or different route of administration, or at different times. The important thing is not the time of administration but having an effective blood level of the P-gp inhibitor.

Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102,918 and other steroids. It is to be understood that additional agents will be found that have the same function and therefore achieve the same outcome; such compounds are also considered to be useful.

The P-gp inhibitors can be administered orally, parenterally, (IV, IM, IM-depo, SQ, SQ-depo), topically, sublingually, rectally, intranasally, intrathecally and by implant.

The therapeutically effective amount of the P-gp inhibitors is from about 0.1 to about 300 mg/kg/day, preferably about 0.1 to about 150 mg/kg daily. It is understood that while a patient may be started on one dose, that dose may have to be varied over time as the patient's condition changes.

When administered orally, the P-gp inhibitors can be administered in usual dosage forms for oral administration as is known to those skilled in the art. These dosage forms include the usual solid unit dosage forms of tablets and capsules as well as liquid dosage forms such as solutions, suspensions and elixirs. When the solid dosage forms are used, it is preferred that they be of the sustained release type so that the P-gp inhibitors need to be administered only once or twice daily. The oral dosage forms are administered to the patient one thru four times daily. It is preferred that the P-gp inhibitors be administered either three or fewer times a day, more preferably once or twice daily. Hence, it is preferred that the P-gp inhibitors be administered in solid dosage form and further it is preferred that the solid dosage form be a sustained release form which permits once or twice daily dosing. It is preferred that what ever dosage form is used, that it be designed so as to protect the P-gp inhibitors from the acidic environment of the stomach. Enteric coated tablets are well known to those skilled in the art. In addition, capsules filled with small spheres each coated to protect from the acidic stomach, are also well known to those skilled in the art.

In addition, the P-gp inhibitors can be administered parenterally. When administered parenterally they can be administered IV, IM, depo-IM, SQ or depo-SQ.

The P-gp inhibitors can be given sublingually. When given sublingually, the P-gp inhibitors should be given one thru four times daily in the same amount as for IM administration.

The P-gp inhibitors can be given intranasally. When given by this route of administration, the appropriate dosage forms are a nasal spray or dry powder as is known to those skilled in the art. The dosage of the P-gp inhibitors for intranasal administration is the same as for IM administration.

The P-gp inhibitors can be given intrathecally. When given by this route of administration the appropriate dosage form can be a parenteral dosage form as is known to those skilled in the art.

The P-gp inhibitors can be given topically. When given by this route of administration, the appropriate dosage form is a cream, ointment or patch. Because of the amount of the P-gp inhibitors needed to be administered the patch is preferred. However, the amount that can be delivered by a patch is limited. Therefore, two or more patches may be required. The number and size of the patch is not important, what is important is that a therapeutically effective amount of the P-gp inhibitors be delivered as is known to those skilled in the art.

The P-gp inhibitors can be administered rectally by suppository as is known to those skilled in the art.

The P-gp inhibitors can be administered by implants as is known to those skilled in the art.

There is nothing novel about the route of administration nor the dosage forms for administering the P-gp inhibitors. Given a particular P-gp inhibitor, and a desired dosage form, one skilled in the art would know how to prepare the appropriate dosage form for the P-gp inhibitor.

It should be apparent to one skilled in the art that the exact dosage and frequency of administration will depend on the particular compounds of the invention administered, the particular condition being treated, the severity of the condition being treated, the age, weight, general physical condition of the particular patient, and other medication the individual may be taking as is well known to administering physicians who are skilled in this art.

Inhibition of APP Cleavage

The compounds of the invention inhibit cleavage of APP between Met596 and Asp597 numbered for the APP695 isoform, or a mutant thereof, or at a corresponding site of a different isoform, such as APP751 or APP770, or a mutant thereof (sometimes referred to as the “beta secretase site”). While not wishing to be bound by a particular theory, inhibition of beta-secretase activity is thought to inhibit production of beta amyloid peptide (A beta). Inhibitory activity is demonstrated in one of a variety of inhibition assays, whereby cleavage of an APP substrate in the presence of a beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compound inhibitors of the invention are known. Representative assay systems are described, for example, in U.S. Pat. Nos. 5,942,400, 5,744,346, as well as in the Examples below.

The enzymatic activity of beta-secretase and the production of A beta can be analyzed in vitro or in vivo, using natural, mutated, truncated, and/or synthetic APP substrates, natural, mutated, truncated and/or synthetic enzyme, and the test compound. The analysis may involve primary or secondary cells expressing native, mutant, and/or synthetic APP and enzyme, animal models expressing native APP and enzyme, or may utilize transgenic animal models expressing the substrate and enzyme. Detection of enzymatic activity can be by analysis of one or more of the cleavage products, for example, by immunoassay, fluorometric or chromogenic assay, HPLC, or other means of detection. Inhibitory compounds are determined as those having the ability to decrease the amount of beta-secretase cleavage products produced in comparison to a control, where beta-secretase mediated cleavage in the reaction system is observed and measured in the absence of inhibitory compounds.

Beta-Secretase

Various forms of beta-secretase enzyme are known, and are available and useful for assay of enzyme activity and inhibition of enzyme activity. These include native, recombinant, and synthetic forms of the enzyme. Human beta-secretase is known as Beta Site APP Cleaving Enzyme (BACE), Asp2, and memapsin 2, and has been characterized, for example, in U.S. Pat. No. 5,744,346 and published PCT patent applications WO98/22597, WO00/03819, WO01/23533, and WO00/17369, as well as in literature publications (Hussain et al., 1999, Mol. Cell. Neurosci. 14:419-427; Vassar et al., 1999, Science 286:735-741; Yan et al., 1999, Nature 402:533-537; Sinha et al., 1999, Nature 40:537-540; and Lin et al., 2000, PNAS USA 97:1456-1460). Synthetic forms of the enzyme have also been described (WO98/22597 and WO00/17369). Beta-secretase can be extracted and purified from human brain tissue and can be produced in cells, for example mammalian cells expressing recombinant enzyme.

Preferred compounds are effective to inhibit 50% of beta-secretase enzymatic activity at a concentration of less than 50 micromolar, preferably at a concentration of 10 micromolar or less, more preferably 1 micromolar or less, and most preferably 10 nanomolar or less.

APP Substrate

Assays that demonstrate inhibition of beta-secretase-mediated cleavage of APP can utilize any of the known forms of APP, including the 695 amino acid “normal” isotype described by Kang et al., 1987, Nature 325:733-6, the 770 amino acid isotype described by Kitaguchi et. al., 1981, Nature 331:530-532, and variants such as the Swedish Mutation (KM670-1NL) (APP-SW), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK disclosed, for example, in WO 00/17369, fragments of APP, and synthetic peptides containing the beta-secretase cleavage site, wild type (WT) or mutated form, e.g., SW, as described, for example, in U.S. Pat. No. 5,942,400 and WO00/03819.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. A useful moiety may be an antigenic epitope for antibody binding, a label or other detection moiety, a binding substrate, and the like.

Antibodies

Products characteristic of APP cleavage can be measured by immunoassay using various antibodies, as described, for example, in Pirttila et al., 1999, Neuro. Lett. 249:21-4, and in U.S. Pat. No. 5,612,486. Useful antibodies to detect A beta include, for example, the monoclonal antibody 6E10 (Senetek, St. Louis, Mo.) that specifically recognizes an epitope on amino acids 1-16 of the A beta peptide; antibodies 162 and 164 (New York State Institute for Basic Research, Staten Island, N.Y.) that are specific for human A beta 1-40 and 1-42, respectively; and antibodies that recognize the junction region of beta-amyloid peptide, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130.

Assay Systems

Assays for determining APP cleavage at the beta-secretase cleavage site are well known in the art. Exemplary assays, are described, for example, in U.S. Pat. Nos. 5,744,346 and 5,942,400, and described in the Examples below.

Cell Free Assays

Exemplary assays that can be used to demonstrate the inhibitory activity of the compounds of the invention are described, for example, in WO00/17369, WO 00/03819, and U.S. Pat. Nos. 5,942,400 and 5,744,346. Such assays can be performed in cell-free incubations or in cellular incubations using cells expressing a beta-secretase and an APP substrate having a beta-secretase cleavage site.

An APP substrate containing the beta-secretase cleavage site of APP, for example, a complete APP or variant, an APP fragment, or a recombinant or synthetic APP substrate containing the amino acid sequence: KM-DA or NL-DA, is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that may be fusion proteins or peptides that contain the substrate peptide and a modification useful to facilitate the purification or detection of the peptide or its beta-secretase cleavage products. Useful modifications include the insertion of a known antigenic epitope for antibody binding; the linking of a label or detectable moiety, the linking of a binding substrate, and the like.

Suitable incubation conditions for a cell-free in vitro assay include, for example: approximately 150 nanomolar to 10 micromolar substrate, approximately 10 to 200 picomolar enzyme, and approximately 0.1 nanomolar to 10 micromolar inhibitor compound, in aqueous solution, at an approximate pH of 4-7, at approximately 37 degrees C., for a time period of approximately 10 minutes to 3 hours. These incubation conditions are exemplary only, and can be varied as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

Cellular Assay

Numerous cell-based assays can be used to analyze beta-secretase activity and/or processing of APP to release A beta. Contact of an APP substrate with a beta-secretase enzyme within the cell and in the presence or absence of a compound inhibitor of the invention can be used to demonstrate beta-secretase inhibitory activity of the compound. Preferably, assay in the presence of a useful inhibitory compound provides at least about 30%, most preferably at least about 50% inhibition of the enzymatic activity, as compared with a non-inhibited control.

In one embodiment, cells that naturally express beta-secretase are used. Alternatively, cells are modified to express a recombinant beta-secretase or synthetic variant enzyme as discussed above. The APP substrate may be added to the culture medium or is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A beta from APP provide a useful means to assay inhibitory activities of the compounds of the invention. Production and release of A beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound inhibitor to demonstrate inhibition of enzymatic activity as compared with a control. Activity of beta-secretase can be measured by analysis of one or more cleavage products of the APP substrate. For example, inhibition of beta-secretase activity against the substrate APP would be expected to decrease release of specific beta-secretase induced APP cleavage products such as A beta.

Although both neural and non-neural cells process and release A beta, levels of endogenous beta-secretase activity are low and often difficult to detect by EIA. The use of cell types known to have enhanced beta-secretase activity, enhanced processing of APP to A beta, and/or enhanced production of A beta are therefore preferred. For example, transfection of cells with the Swedish Mutant form of APP (APP-SW); with APP-KK; or with APP-SW-KK provides cells having enhanced beta-secretase activity and producing amounts of A beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound inhibitor, the amount of A beta released into the medium and/or the amount of CTF99 fragments of APP in the cell lysates is reduced as compared with the control. The cleavage products of APP can be analyzed, for example, by immunoassays with specific antibodies, as discussed above.

Preferred cells for analysis of beta-secretase activity include primary human neuronal cells, primary guinea pig neuronal cells, primary transgenic animal neuronal cells where the transgene is APP, and other cells such as those of a stable H4 neuroblastoma cell line expressing APP, for example, APP-SW.

In vivo Assays: Animal Models

Various animal models can be used to analyze beta-secretase activity and/or processing of APP to release A beta, as described above. For example, transgenic animals expressing APP substrate and/or beta-secretase enzyme can be used to demonstrate inhibitory activity of the compounds of the invention. Certain transgenic animal models have been described, for example, in U.S. Pat. Nos. 5,877,399; 5,612,486; 5,387,742; 5,720,936; 5,850,003; 5,877,015, and 5,811,633, and in Ganes et al., 1995, Nature 373:523. Preferred are animals that exhibit characteristics associated with the pathophysiology of AD. Administration of the compound inhibitors of the invention to the transgenic mice described herein provides an alternative method for demonstrating the inhibitory activity of the compounds. Administration of the compounds in a pharmaceutically effective carrier and via an administrative route that reaches the target tissue in an appropriate therapeutic amount is also preferred.

Inhibition of beta-secretase mediated cleavage of APP at the beta-secretase cleavage site and of A beta release can be analyzed in these animals by measure of cleavage fragments in the animal's body fluids such as cerebral spinal fluid, plasma or tissues. Analysis of brain tissues for A beta deposits or plaques may also suggest inhibition of beta-secretase activity.

On contacting an APP substrate with a beta-secretase enzyme in the presence of an inhibitory compound of the invention and under conditions sufficient to permit enzymatic mediated cleavage of APP and/or release of A beta from the substrate, the compounds of the invention are effective to reduce beta-secretase-mediated cleavage of APP at the beta-secretase cleavage site and/or effective to reduce released amounts of A beta. Where such contacting is the administration of the inhibitory compounds of the invention to an animal model, for example, as described above, the compounds are effective to reduce A beta deposition in brain tissues of the animal, and to reduce the number and/or size of beta amyloid plaques. Where such administration is to a human subject, the compounds are effective to inhibit or slow the progression of disease characterized by reduced amounts of A beta, to slow the progression of AD, and/or to prevent onset or development of AD in a patient at risk for the disease.

Unless defined otherwise, all scientific and technical terms used herein have the same meaning as commonly understood by one of skill in the art to which this invention belongs. All patents and publications referred to herein are hereby incorporated by reference for all purposes.

The present invention may be better understood with reference to the following examples. These examples are intended to be representative of specific embodiments of the invention, and are not intended as limiting the scope of the invention.

### PREPARATIVE EXAMPLES

### A. Preparation of 7-(2,2-Dimethyl-propyl)-2-piperidin-1-yl-3,4-dihydro-2H-naphthalen-1-one

Copper(II) bromide (1.05 g, 4.70 mmol) is added to a refluxing solution of 7-(2,2-dimethyl-propyl)-3,4-dihydro-2H-naphthalen-1-one (1.0 g, 4.62 mmol) in 1:1 ethyl acetate/chloroform. After refluxing for about 1 hr, the mixture is cooled, filtered (Celite) to remove copper salts, washed with water and brine, dried (MgSO₄) and concentrated to afford 2-bromo-7-(2,2-dimethyl-propyl)-3,4-dihydro-2H-naphthalen-1-one. This material is immediately dissolved in THF (50 mL), triethylamine (1.3 mL, 9.32 mmol) is added followed by piperidine (0.51 mL, 5.15 mmol) and the resulting mixture is stirred at ambient temperature overnight. The reaction mixture is concentrated, dissolved in CH₂Cl₂ and washed with water and brine, dried (MgSO₄) and concentrated to yield 7-(2,2-dimethyl-propyl)-2-piperidin-1-yl-3,4-dihydro-2H-naphthalen-1-one.

### B. Preparation of 7-(2,2-Dimethyl-propyl)-2-piperidin-1-yl-1,2,34-tetrahydro-naphthalen-1-ylamine

7-(2,2-Dimethyl-propyl)-2-piperidin-1-yl-3,4-dihydro-2H-naphthalen-1-one (0.45 g, 1.50 mmol), hydroxylamine hydrochloride (0.11 g, 1.58 mmol), and pyridine (1.28 mL, 15.8 mmol) in ethanol (25 mL) are refluxed for about 1.5 hr, cooled and concentrated to dryness. The residue is partitioned between ethyl acetate and water, the organics are washed with brine, dried (MgSO₄) and concentrated to give a mixture of oxime isomers. These are dissolved in ethanol and hydrogenated at 45 psi in the presence of Raney nickel (0.3 g, w/w). After about 16 hrs, the reaction is filtered and concentrated to afford 7-(2,2-dimethyl-propyl)-2-piperidin-1-yl-1,2,3,4-tetrahydro-naphthalen-1-ylamine.

### Example 1

### Preparation of N-{1-(3,5-Difluoro-benzyl)-3-[7-(2,2-dimethyl-propyl)-2-piperidin-1-yl-1,2,3,4-tetrahydro-naphthalen-1-ylamino]-2-hydroxy-propyl}-acetamide

tert-Butyl(S)-2-(3,5-difluorophenyl)-1-((S)-oxiran-2-yl)ethylcarbamate (0.36 g, 1.20 mmol)) and 7-(2,2-dimethyl-propyl)-2-piperidin-1-yl-1,2,3,4-tetrahydro-naphthalen-1-ylamine (0.30 g, 1.00 mmol) in isopropanol are refluxed for about 16 hrs, cooled and concentrated onto silica gel. Chromatography using 5-15% methanol/ethyl acetate removes the unreacted epoxide and affords {1-(3,5-difluoro-benzyl)-3-[7-(2,2-dimethyl-propyl)-2-piperidin-1-yl-1,2,3,4-tetrahydro-naphthalen-1-ylamino]-2-hydroxy-propyl}-carbamic acid tert-butyl ester. This is dissolved in dioxane (3 mL) and treated with 4N HCl/dioxane. The mixture is stirred for about 18 hrs at ambient temperature and concentrated to give the hydrochloride salt of 3-amino-4-(3,5-difluoro-phenyl)-1-[7-(2,2-dimethyl-propyl)-2-piperidin-1-yl-1,2,3,4-tetrahydro-naphthalen-1-ylamino]-butan-2-ol. This crude amine is dissolved in CH₂Cl₂ (10 mL) and cooled in ice. N-Methylmorpholine (0.66 mmol, 6.00 mmol), acetic acid (0.057 mL, 1.00 mmol), HOBT (0.15 g, 1.11 mmol) and EDC (0.21 g, 1.10 mmol) are added sequentially and the reaction mixture is allowed to warm to room temperature over about 16 hrs. The reaction is concentrated, partitioned between ethyl acetate and water, the organics are washed with brine, dried (MgSO₄) and concentrated. The crude product is purified by column chromatography using 5-20% methanol/ethyl acetate to give N-{1-(3,5-difluoro-benzyl)-3-[7-(2,2-dimethyl-propyl)-2-piperidin-1-yl-1,2,3,4-tetrahydro-naphthalen-1-ylamino]-2-hydroxy-propyl}-acetamide.

### Biological Examples

### Example A

Cell Free BACE1 Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds. The substrate is Biotin-GLTNIKTEEISEISY-EVEFRC[oregon green]KK [SEQ ID NO: 1]. The enzyme (0.1 nanomolar) and test compounds (0.00002-200 micromolar) are incubated in pre-blocked, low affinity, black plates (384 well) at RT for 30 minutes. The reaction is initiated by addition of 150 nanomolar substrate to a final volume of 30 microliter per well. The final assay conditions are: 0.00002-200 micromolar compound inhibitor; 0.1 molar sodium acetate (pH 4.5); 150 nanomolar substrate; 0.1 nanomolar soluble beta-secretase; 0.001% Tween 20, and 2% DMSO. The assay mixture is incubated for 3 hours at 37 degrees C., and the reaction is terminated by the addition of a saturating concentration of immunopure streptavidin (0.75 micromolar). After incubation with streptavidin at room temperature for 15 minutes, fluorescence polarization is measured, for example, using a PerkinElmer Envision (Ex485 nm/Em530 nm). The activity of the beta-secretase enzyme is detected by changes in the fluorescence polarization that occur when the substrate is cleaved by the enzyme. Incubation in the presence of compound inhibitor demonstrates specific inhibition of beta-secretase enzymatic cleavage of its synthetic APP substrate. In this assay, preferred compounds of the invention exhibit an IC₅₀ of less than 50 micromolar. More preferred compounds of the invention exhibit an IC₅₀ of less than 10 micromolar. Even more preferred compounds of the invention exhibit an IC₅₀ of less than 5 micromolar. Even more preferred compounds of the invention exhibit an IC₅₀ of less than 100 nanomolar. Even more preferred compounds of the invention exhibit an IC₅₀ of less than 10 nanomolar.

### Example B

Inhibition of Beta-Secretase Activity—Cellular Assay

An exemplary assay for the analysis of inhibition of beta-secretase activity utilizes the H4 human neuroblastoma cell line (ATCC Accession No. CRL-1573) containing the naturally occurring double mutation Lys651Met52 to Asn651Leu652 (numbered for APP751), commonly called the Swedish mutation and shown to overproduce A beta (Citron et al., 1992, Nature 360:672-674), as described in U.S. Pat. No. 5,604,102.

The cells are incubated in the presence/absence of the inhibitory compound (applied from DMSO stock) at the desired concentration, generally up to 30 □M at a final concentration of <5% DMSO. At the end of the treatment period, conditioned media is analyzed for beta-secretase activity, for example, by analysis of cleavage fragments. A beta can be analyzed by immunoassay, using specific detection antibodies. The enzymatic activity is measured in the presence and absence of the compound inhibitors to demonstrate specific inhibition of beta-secretase mediated cleavage of APP substrate.

For example, the assay may be conducted in two days by plating cells in the morning of day 1 (30,000 cells/well plating density, H4swe) and incubating at 37 C. for approx. 6 hrs. In the afternoon of day 1 the cells are washed once with media or PBS to remove accumulated AB. Fresh media is added and the compound of the invention is then added to each well (95□l media+5□l20× compound stock in 10% DMSO). The cells are incubated overnight at 37 C. in 5% CO₂.

A set of 96-well Nunc immuno-sorp plates is coated with A□ capture antibody (6E10); each well is coated with 50 □l of a 1 mg/ml 6E10 prepared in Carbonate/Bicarbonate coating buffer. The Carbonate/Bicarbonate coating buffer contained 500 ml purified water at pH 9.5, 0.8 g Na₂CO₃ and 1.5 g NaHCO₃. Plates are sealed and incubated overnight at 4° C.

On day 2, the 6E10 coated plates are brought to room temperature in 15-30 min. and washed with 0.05% PBST 3-4 times using a plate washer. 200 □l/well of 1% milk in PBST are added to block non-specific binding of subsequently added conditioned media or AD standards.

The plates are covered, and incubated at room temperature for at least 1h. The plates are washed with PBST 3-4 times to remove block. Cell conditioned media from the H4 cells (pretreated overnight with compound as described above) or Aβ standards are added to wells (50 ul/well, 2h, dark, room temperature). An MTT assay is optionally performed on the cells (2h incubation, 37° C.).

The ELISA plates are washed 3-4 times with PBST. Biotinylated reporter Aβ antibody is added (50 □l/well (4G8-biotinylated 1:5000 in PBST), 1-2 hr, room temperature), dark) followed by washing 3-4 times with PBST. Streptavidin-HRP (1:10,000 in PBST) is added (50 □l/well) and the plates are incubated (30 min-1 hr, room temperature, dark). The plates are washed 3-4 times with PBST. 50 μl/well of TMB-peroxidase substrate are added at room temperature and incubation is carried out at room temperature until desired blue color develops. The reaction is stopped by adding 50□l/well of 0.09M H₂SO₄. Color becomes yellow. Reading at 450 nm on Spectramax is performed.

### Example C

Guinea Pig Neuronal Culture Assay

Mixed cortical and hippocampal neurons are isolated from guinea pig embryos and cultured in vitro. In an exemplary procedure, at about embryonic day 25, a guinea pig is anaesthetized in isoflurane chamber and decapitated. The uterus with the embryos is removed and placed into 25 mL cold Hibernate E in a 50 mL tube on wet ice. In a petri dish on a cold plate (4° C.) containing just enough Hibernate E under a dissecting microscope, the skull is peeled back and the brain and cerebellum removed. Separation of the hemispheres is followed by removal of the midbrain. The meninges are stripped away from the cortices. The cortical tissue is placed in Hibernate E/B27 in a 15 ml tube on wet ice, pooling tissue from all the embryos (3-6 embryos in 10 mL Hib E/B27). B27 (50× solution Gibco BRL*17504-044) is added in a 1:50 ratio to the Hibernate E medium.

All of the Hibernate E/B27 is removed from the tube and transferred to a new 15 mL tube for trituration. 5 ml of 2 mg/ml Papain solution is added to the tissue in tube for 25 min. at 37° C. If more than 6 embryos of tissue pooled use 10 mL of Papain solution. 1 ml of 10 mg/ml Papain (Roche 0108014)+4 ml Hibernate E with no B27 is warmed for 5 min. at 37° C. and filtered through a 0.2 □m syringe filter.

A first trituration is performed with 2 ml Hibernate E/B27 and 20 μL of 1 mg/ml DNAse (Sigma D4263). Tissue is triturated 10-15 times through a flame-polished Pasteur pipet after removal of the papain solution. 1-2 additional trituration steps are performed, 10-15 triturations each time with 2 mL Hibernate E/B27, each time decreasing size of the pipet opening with a flame. The supernatants are filtered through a 100 □m strainer (Falcon 352350) and spun down for 5 min. at 250×g. The pellet is resuspended with 10 mls plating medium containing B27 (50× solution, Gibco BRL 17504-044) in a 1:50 ratio, 0.5 mM Glutamine (200 mM Gibco BRL 25030-081) in a 1:400 ratio, Invitrogen 15240062 antibiotic-antimycotic (100×), and 250M glutamate in a 1:1000 ratio from 25 mM stock in 50% 0.1 M HCl/50% Neurobasal (the glutamate is only used in the initial plating medium). The cells are plated on poly-D-lysine 24-well plates (Biocoat 356414), in a concentration of 1.5×10⁵ cells/cm²=6×10⁵ cells/mL, adding 500 □L/well.

Neurons are re-fed every 4-5 days after culturing in a culture medium with 500 □l/well Neurobasal/B27+glutamine+P/S/FZ without glutamate. Baseline samples (300 □l) are taken from each well on DIV 15 (3-4 days after the last refeed), transferred to a 96-well plate and frozen at −20° C. until assayed.

Diluted samples of the compounds of the invention are prepared from 10 mM stocks in 100% DMSO in a 96-well plate with each drug in its own row of 12 wells, the resulting concentrations in the wells being 10, 3, 1, 0.3, 0.1, 0.03, 0.01, 0.003, 0.001, 0.0003, 0.0001 and 0.00003 mM in DMSO. Each well is diluted 1:10 in re-fed media. The remaining media is aspirated from all cells and replaced with 500 μL fresh media. The compounds are added to duplicate wells at 1:100 dilution (by adding 5 μL per well). Final compound concentrations range from 10 □M to 100 pM. After 3-4 days*, samples are removed from each well (300 □l) and transferred to a 96-well plate. The samples are subjected to freeze treatment at −20° C. until assaying.

Assay for cytotoxicity using the MTS assay (Promega G3581) was performed on each well with MTS one solution reagent/well (10 μl), including wells of a plate with only medium for background color subtraction, for 2 hrs at 37° C. 100 □l from each well were transferred into a new 96-well plate and 2 medium only/MTS reagent wells were included for background subtraction. Readings were made at 490 nm. For sample assaying, samples were thawed at R.T. and diluted 1:2 using ELISA sample/blocking buffer PBST+1% BSA

*For total Aβ measurement, as short as 24 h is sufficient to measure A□, though 3 days in culture will produce a much more robust signal.

For C-terminal specific assays (i.e. Aβ 1-38/1-40/1-42), a full 4 days are required before collecting media, as signal (especially Aβ 1-42) is quite low.

### Example D

Inhibition of Beta-Secretase in Animal Models of AD

Various animal models can be used to screen for inhibition of beta-secretase activity. Examples of animal models useful in the invention include, but are not limited to, mouse, guinea pig, dog, and the like. The animals used can be wild type, transgenic, or knockout models. In addition, mammalian models can express mutations in APP, such as APP695-SW and the like described herein. Examples of transgenic non-human mammalian models are described in U.S. Pat. Nos. 5,604,102, 5,912,410 and 5,811,633.

Tg2576 mice, prepared as described in Hsiao et al. 1996 are useful to analyze in vivo suppression of A beta release in the presence of putative inhibitory compounds. Tg2576 mice are administered compound formulated in vehicle, such as 20% DMSO:20% ethanol: 60% polyethlylene glycol. The mice are dosed with compound (100-300 mg/kg; preferably 1-100 mg/kg). After time, e.g., 3-10 hours, the animals are sacrificed, and brains removed for analysis.

Transgenic animals are administered an amount of the compound inhibitor formulated in a carrier suitable for the chosen mode of administration. Control animals are untreated, treated with vehicle, or treated with an inactive compound. Administration can be acute, i.e., single dose or multiple doses in one day, or can be chronic, i.e., dosing is repeated daily for a period of days, weeks or months. Beginning at time 0, brain tissue or cerebral spinal fluid or plasma is obtained from selected animals and analyzed for the presence of APP cleavage peptides, including A beta, for example, by immunoassay using specific antibodies for A beta detection. At the end of the test period, animals are sacrificed and brain tissue or cerebral spinal fluid or plasma is analyzed for the presence of A beta, sAPP□ and/or beta-amyloid plaques. The tissue is also analyzed for signs of degeneration.

Animals administered the compound inhibitors of the invention are expected to demonstrate reduced A beta in brain tissues or cerebral spinal fluids or plasma and/or reduced beta amyloid plaques in brain tissue, as compared with non-treated controls.

### Example E

Inhibition of A Beta Production in Human Patients

Patients suffering from Alzheimer's Disease (AD) demonstrate an increased amount of A beta in the brain. AD patients are administered an amount of the compound inhibitor formulated in a carrier suitable for the chosen mode of administration. Administration is repeated daily for the duration of the test period. Beginning on day 0, cognitive and memory tests are performed, for example, once per month.

Patients administered the compound inhibitors are expected to demonstrate slowing or stabilization of disease progression as analyzed by changes in one or more of the following disease parameters: A beta present in CSF or plasma; brain or hippocampal volume; A beta deposits in the brain; amyloid plaque in the brain; and scores for cognitive and memory function, as compared with control, non-treated patients.

### Example F

Prevention of A Beta Production in Patients at Risk for AD

Patients predisposed or at risk for developing AD are identified either by recognition of a familial inheritance pattern, for example, presence of the Swedish Mutation, and/or by monitoring diagnostic parameters. Patients identified as predisposed or at risk for developing AD are administered an amount of the compound inhibitor formulated in a carrier suitable for the chosen mode of administration. Administration is repeated daily for the duration of the test period. Beginning on day 0, cognitive and memory tests are performed, for example, once per month.

Patients administered the compound inhibitors are expected to demonstrate slowing or stabilization of disease progression as analyzed by changes in one or more of the following disease parameters: A beta present in CSF or plasma; brain or hippocampal volume; amyloid plaque in the brain; and scores for cognitive and memory function, as compared with control, non-treated patients.

The invention has been described with reference to various specific and preferred embodiments and techniques. However, it should be understood that many variations and modifications may be made while remaining within the spirit and scope of the invention.

1. A compound of the formula I 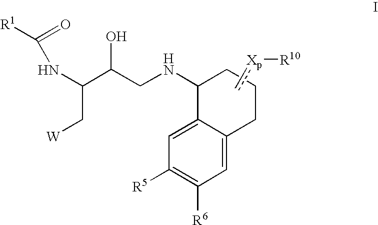
wherein
W is selected from the group consisting of (i) C₆-C₁₀ aryl optionally substituted with one to three substituents each independently selected from H, halogen, CN, NO₂, OH, O—C₁-C₈ alkyl, NH₂, NH—C₁-C₈alkyl, N(C₁-C₈alkyl)₂, NHCO—C₁-C₈alkyl, and CONH—C₁-C₈alkyl, and (ii) C₅-C₁₀ heteroaryl optionally substituted with one to three substituents each independently selected from H, halogen, CN, NO₂, OH, O—C₁-C₈ alkyl, NH₂, NH—C₁-C₈ alkyl, N(C₁-C₈ alkyl)₂, NHCO—C₁-C₈ alkyl, and CONH—C₁-C₈ alkyl,
R¹ is C₁-C₆ alkyl, optionally substituted with up to three fluoro atoms;
R⁵ and R⁶ are each independently selected from the group consisting of H, C₁-C₈ alkyl, C₂-C₈ alkenyl, C₃-C₈ alkynyl, C₃-C₅ cycloalkyl, 5-10 membered heteroaryl, and C₆-C₁₄ aryl;
p is from 0 to 1, [symbol]
represents a single or double bond, with the proviso that if [symbol] is a double bond, X is N, C(C₁-C₆ alkyl), or O, with the proviso that if X is O and p is 1, R¹⁰ is absent; and with the proviso that if [symbol] is a single bond, X is C₀-C₆ alkylene, C(═C₁-C₆ alkylidene), C(—N—O—C₁-C₆ alkyl), C(═N—C₁-C₆ alkyl), C═O, NH, NC₁-C₆ alkyl, or O; and
R¹⁰ is selected from the group consisting of H, C₁-C₆ alkyl, —OH, —O—C₁-C₆ alkyl, —CO—C₁-C₆ alkyl, —COO—C₁-C₆ alkyl, —NH—C₁-C₆ alkyl, —N(C₁-C₆ alkyl)-C₁-C₆ alkyl, —CO—N(C₁-C₆ alkyl)-C₁-C₆ alkyl, 5-10 membered heteroaryl, 5-10-membered heterocycloalkyl, C₆-C₁₄ aryl, S(O)_{q}C₁-C₆ alkyl, and S(O)_{q}C₆-C₁₀ aryl.

2. The compound of claim 1 wherein R¹ is CH₃.

3. The compound of claim 1 wherein R⁵ is neopentyl or t-butyl.

4. The compound of claim 1 wherein [symbol] represents a single bond and X is selected from the group consisting of C(═C₁-C₆ alkylidene), C(═N—O—C₁-C₆ alkyl), C(═N—C₁-C₆ alkyl), C═O, NH, NC₁-C₆ alkyl, and O.

5. The compound of claim 1 wherein [symbol] represents a double bond and X is selected from the group consisting of N and C(C₁-C₆ alkyl).

6. The compound of claim 1 wherein R¹⁰ is selected from the group consisting of H, C₁-C₆ alkyl, —OH, —O—C₁-C₆ alkyl, —CO—C₁-C₆ alkyl, —COO—C₁-C₆ alkyl, —NH—C₁-C₆ alkyl, —CO—N(C₁-C₆ alkyl)-C₁-C₆ alkyl, 5-10 membered heteroaryl, 5-10-membered heterocycloalkyl, and C₆-C₁₄ aryl.

7. The compound of claim 1 wherein the compound is a compound of formula Ia: 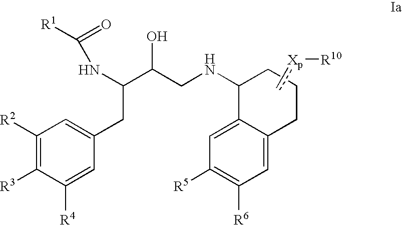
wherein R², R³, R⁴ are each independently selected from the group consisting of H, halogen, CN, NO₂, OH, O—C₁-C₈ alkyl, NH₂, NH—C₁-C₈ alkyl, N(C₁-C₈ alkyl)₂, NHCO—C₁-C₈ alkyl, and CONH—C₁-C₈.

8. The compound of claim 8 wherein R² is fluoro.

9. The compound of claim 8 wherein R⁴ is fluoro.

10. The compound of claim 8 wherein the compound is a compound of the formula Iaa 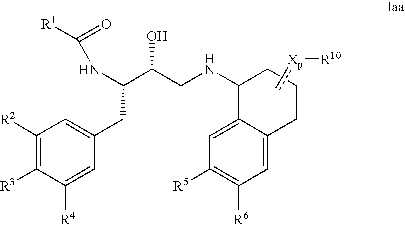
or an enantiomer of the compound of the formula Iaa.

11. The compound of claim 13, wherein the compound is the optical isomer Iaa.

12. The compound of claim 1, wherein the compound is selected from the group consisting of:
N-{1-(3,5-Difluoro-benzyl)-3-[7-(2,2-dimethyl-propyl)-2-piperidin-1-yl-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-phenyl-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-(1H-imidazol-2-yl)-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-morpholin-4-yl-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-methylamino-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-methoxy-1,2,3,4-tetrahydro-naphthalen-1-ylamino]-2-hydroxy-propyl}-acetamide;
1-[3-Acetylamino-4-(3,5-difluoro-phenyl)-2-hydroxy-butylamino]-7-(2,2-dimethyl-propyl)-1,2,3,4-tetrahydro-naphthalene-2-carboxylic acid methyl ester;
1-[3-Acetylamino-4-(3,5-difluoro-phenyl)-2-hydroxy-butylamino]-7-(2,2-dimethyl-propyl)-1,2,3,4-tetrahydro-naphthalene-2-carboxylic acid dimethylamide;
N-[3-[2-Acetyl-7-(2,2-dimethyl-propyl)-1,2,3,4-tetrahydro-naphthalen-1-ylamino]-1-(3,5-difluoro-benzyl)-2-hydroxy-propyl]-acetamide;
Dimethyl-carbamic acid 1-[3-acetylamino-4-(3,5-difluoro-phenyl)-2-hydroxy-butylamino]-7-(2,2-dimethyl-propyl)-1,2,3,4-tetrahydro-naphthalen-2-yl ester;
Carbonic acid 1-[3-acetylamino-4-(3,5-difluoro-phenyl)-2-hydroxy-butylamino]-7-(2,2-dimethyl-propyl)-1,2,3,4-tetrahydro-naphthalen-2-yl ester methyl ester;
N-{1-(3,5-Difluoro-benzyl)-3-[7-(2,2-dimethyl-propyl)-2-(1,3,3-trimethyl-ureido)-1,2,3,4-tetrahydro-naphthalen-1-ylamino]-2-hydroxy-propyl}-acetamide;
[1-[3-Acetylamino-4-(3,5-difluoro-phenyl)-2-hydroxy-butylamino]-7-(2,2-dimethyl-propyl)-1,2,3,4-tetrahydro-naphthalen-2-yl]-methyl-carbamic acid methyl ester;
N-{1-(3,5-Difluoro-benzyl)-3-[7-(2,2-dimethyl-propyl)-2-(1-methoxyimino-ethyl)-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-(1-methoxy-ethyl)-1,2,3,4-tetrahydro-naphthalen-1-ylamino]-2-hydroxy-propyl}-acetamide;
N-[3-[2-Acetylamino-7-(2,2-dimethyl-propyl)-1,2,3,4-tetrahydro-naphthalen-1-ylamino]-1-(3,5-difluoro-benzyl)-2-hydroxy-propyl]-acetamide;
N-{1-(3,5-Difluoro-benzyl)-3-[7-(2,2-dimethyl-propyl)-2-methanesulfonylamino-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-(3-methyl-3H-imidazol-1-4-ylmethylene)-1,2,3,4-tetrahydro-naphthalen-1-ylamino]-2-hydroxy-propyl}-acetamide;
N-{1-(3,5-Difluoro-benzyl)-3-[7-(2,2-dimethyl-propyl)-2-sulfamoyl-1,2,3,4-tetrahydro-naphthalen-1-ylamino]-2-hydroxy-propyl}-acetamide;
N-[3-[7-(2,2-Dimethyl-propyl)-2-morpholin-4-yl-1,2,3,4-tetrahydro-naphthalen-1-ylamino]-1-(4-fluoro-pyridin-2-ylmethyl)-2-hydroxy-propyl]-acetamide; and
1-[3-Acetylamino-4-(2,6-difluoro-pyrimidin-4-yl)-2-hydroxy-butylamino]-7-(2,2-dimethyl-propyl)-1,2,3,4-tetrahydro-naphthalene-2-carboxylic acid dimethylamide.

13. A pharmaceutical composition comprising the compound of claim 1 and a pharmaceutically acceptable carrier.

14. A method for preventing or delaying the onset of Alzheimer's disease, preventing or delaying the onset of Alzheimer's disease in patients who would otherwise be expected to progress from mild cognitive impairment to Alzheimer's disease, or preventing potential consequences of cerebral amyloid angiopathy such as single and recurrent lobar hemorrhages, the method comprising administering to a patient in need of such treatment a compound of claim 1 or a pharmaceutically acceptable salt thereof.
